(19) United States
(12) Patent Application Publication
(10) Pub. No.: US 2009/0137369 A1
(43) Pub. Date: May 28, 2009

(54) METHOD AND APPARATUS FOR ENABLING AND MONITORING THE MOVEMENT OF HUMAN LIMBS

(76) Inventors: Thomas P. Branch, (US); Alex Sattler, (US); Eric Kenneth Branch, Weston, FL (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/193,610

(22) Filed: Aug. 18, 2008

Related U.S. Application Data
(63) Continuation-in-part of application No. 11/065,432, filed on Feb. 24, 2005, now abandoned.

(51) Int. Cl. A61F 5/00; A63B 21/008

(52) U.S. Cl. 482/112; 602/32

(57) ABSTRACT

An orthotic apparatus for use in providing improved range of motion is provided which allows the amount of stretch to be hydraulically powered and measured by the device, but controlled by the user. Because the apparatus accurately calculates the amount of stretch, the user, together with the user's physician and therapist, can develop a rehabilitation plan based on accurate measurements. Progress is based on tangible results rather than the user's ability to tolerate pain. This knowledge provides the incentive the user needs to work toward and achieve the user's goal.

### CROSS-REFERENCE TO RELATED APPLICATIONS

This application is a continuation-in-part and claims the full benefit and priority of pending prior application Ser. No. 11/065,432, filed Feb. 24, 2005 and entitled APPARATUS FOR ENABLING THE MOVEMENT OF HUMAN LIMBS AND METHOD FOR USING SAME, which itself claims the priority and benefit of application Ser. No. 10/147,434, filed May 15, 2002 and entitled APPARATUS FOR ENABLING THE MOVEMENT OF HUMAN LIMBS AND METHOD FOR USING SAME, which itself claims the priority and benefit of application No. 60/291,244, filed May 15, 2001 and entitled APPARATUS FOR ENABLING THE MOVEMENT OF HUMAN LIMBS AND METHOD FOR USING SAME, and incorporates both applications by reference. The full benefit and priority of all the above-referenced applications are claimed for the present application.

### FIELD OF THE INVENTION

The present invention relates generally to an orthotic apparatus for enabling the full normal motion of a joint as an alternative to surgical manipulation.

### BACKGROUND OF THE INVENTION

The number one complication of a joint injury is loss of motion. The loss of motion is often due to an excess production of fibrous tissue within the joint called arthrofibrosis. Arthrofibrosis is both a mechanical and a biological process, which results in loss of motion of a joint.

Synovial cells make up the lining of a joint. These cells are the source of the problem called arthrofibrosis. The synovial cells transform themselves into fibroblasts upon exposure to cytokines and growth factors produced by damaged vascular endothelium. Sudden increases in range of motion produced by intermittent vigorous physical therapy or intra-operative manipulation cause bleeding within the joint further exposing the synovial cells to the cytokines and growth factors which cause arthrofibrosis.

The current methods for gaining range of motion in joints with early or late arthrofibrosis include vigorous physical therapy, specialized splints, continuous passive motion machines and surgical manipulation under anesthesia. Unfortunately, vigorous physical therapy and surgical manipulation under anesthesia have a high failure rate associated with peri-articular bleeding and the resultant progression of arthrofibrosis. Continuous passive motion machines are not effective as they spend most of the time in the middle range of motion of the joint and not focused on stretching at end range of motion.

The current specialized splints include serial casting, Dynasplint and the Joint Active System, on which the invention will provide personal opinions. All of these splints enclose the limb segment proximal and distal to the joint that needs to be stretched. Furthermore, the Dynasplint allows for only a low load stretching process. The Joint Active Systems devices allow for higher loads to be placed at the joint but at the expense of increased pressure at the limb segments proximal and distal to the joint. The loads used by the Joint Active Systems are low in intensity. Serial casting splints are not removable by the patient and have limited adjustability to change the load placed at the joint. Due to the splint design of these devices energy is trapped within the structure of the splints during the stretching process. As a result there is an unpredictable variation in load seen by the joint during the stretching process. This ‘unpredictability’ creates a sense of unease in the patient using the device to gain range of motion. None of these devices produce a load high enough to assure that for every degree the device moves the joint moves the same amount. Finally, none of these devices allow for an instantaneous or quick release of the load applied to the joint.

There is a need to produce an orthotic device for the treatment of arthrofibrosis, which can stretch the joint into full normal end range of motion in a predictable, consistent and reliable fashion. This device should be rigid enough to not allow the storage of energy within its structure. Furthermore, it should be able to produce a load at the joint high enough to assure that for every degree the device moves the joint moves the same amount. Finally, this orthotic device should allow for instantaneous or quick release of the load applied to the joint.

### BRIEF SUMMARY OF THE INVENTION

The orthotic device according to the present invention (a.k.a. “inventive device” allows the user to achieve full normal flexion of the knee or ankle while maintaining absolute control of the process. Furthermore, the inventive device produces a sufficient load at the knee or ankle such that for every degree the inventive device moves the knee or ankle moves the same degree. Furthermore, this device allows for measured progress in terms of distance of the heel to the buttock which is the most reliable measure of knee flexion.

The inventive device provides knee flexion to the point where the heel touches the buttock of the patient. This can amount to well over 145 degrees depending upon the patients normal anatomy. The load is applied to the bottom of the foot and the lower back/buttock region of the patient. These areas are used to increased pressure as opposed to the skin on the anterior aspect of the shin or the thigh. Amount of stretch is patient controlled with a hydraulic hand pump and an instantaneous or quick release mechanism which can stop and reverse the load applied to the joint at any moment. Unlike other range of motion devices; the inventive device provides a very high load to a joint in tiny increments, which helps stretch soft tissue without tearing it causing more vascular re-injury. The inventive device is operated hydraulically and does not rely on any electrically powered parts. The inventive device is solid, sturdy and safe.

By making the ankle pivot of the inventive device stationary and providing a heel lock feature, a particular motion of the device transfers load to the ankle causing the ankle to dorsiflex as the knee flexes. When the knee has normal range of motion all of the load is transferred to the ankle and ankle dorsiflexion is achieved in a similar fashion to knee flexion.

The inventive device is believed to be the best and only non-operative method for regaining full flexion in the most difficult patients following any type of knee or ankle injury or surgery.

In a follow up study of 96 patients who failed the use of traditional methods to regain knee flexion post-operatively (including surgery), the use of the inventive device was successful in regaining functional knee flexion in 95% and full knee flexion in greater than 90%.

The amount of stretch is hydraulically powered with up to 30 times more torque at the knee than any other range of motion product on the market. This high powered stretch is fully controlled by the patient. Heel to buttock measurement insures an easy and accurate day to day evaluation of the patient's progress. This progress is based on tangible results rather than the ability to tolerate pain. This knowledge provides the incentive needed to work toward and achieve goals.

Operation is as follows. The patient sits in the device 4-8 times per day using the hydraulic pump to pull the knee into flexion for 1 to 5 minutes of stretch with an equal amount of time spent in a relaxed position for joint recovery for a total of 15 minutes per session. At the end of the session the position of the foot pedal on the measuring tape is noted and helps to determine the goals for the next session. The goals for each session are to stretch the soft tissues causing the restriction in range of motion of the knee without tearing these structures causing vascular re-injury. The physician must direct this treatment protocol as there are distinct contra-indications for the use of this device, e.g. restricted ankle range of motion, restricted hip range of motion, presence of a total hip arthroplasty or a total ankle arthroplasty, or a technical or mechanical issue restricting range of motion of the knee. All of these contra-indications are of great concern considering that this device can develop from less than 1 ft-lb of torque up to 750 ft-lbs of torque at the knee.

When using this device for ankle dorsiflexion it is assumed that knee flexion is normal. The ankle pivot on the foot pedestal is fixed in the appropriate position so that as the knee is flexed the load is applied to the ankle causing ankle dorsiflexion. The protocol is similar to that described for the knee.

Therefore, it is an object of the present invention to provide an improved orthotic device.

It is a further aspect of the present invention to provide an improved orthotic device which is easy to operate.

It is a further aspect of the present invention to provide an improved orthotic device which is simple in design.

It is a further aspect of the present invention to provide an improved orthotic device which is readily stored.

It is a further aspect of the present invention to provide an improved orthotic device which compiles data relating to its use.

Other objects, features, and advantages of the present invention will become apparent upon reading the following detailed description of the preferred embodiment of the invention when taken in conjunction with the drawing and the appended claims.

### BRIEF DESCRIPTION OF THE DRAWING(S)

Having thus described the invention in general terms, reference will now be made to the accompanying drawings, which are not necessarily drawn to scale, and wherein:

FIG. 1 is an overall pictorial view of the apparatus 10 according to the present invention.

FIG. 2 is a side elevational view of the apparatus 10 according to the present invention, having one (leftward) end positioned atop a supporting surface 9. The other (rightward) end is shown as if suspended in air, but in fact this end is supported a distance height “H” above the supporting surface 9, by use of a separate chair, which is not shown in this figure.

FIG. 3 is a partial pictorial view of the head end 22 of the frame 20 of the apparatus 10, being supported by a cross member 8 of a chair 5

FIG. 4 is a partial pictorial view of the combination of the apparatus 10 with a chair 5, with a user 1000 seated in the chair and having the user's right leg positioned atop the apparatus 10. The user's right hand 1003 is holding the manual pump member 60, and the user's right hand thumb is positioned adjacent the switch 1000.

FIG. 5 is a partial pictorial view of the right hand 1003 of a user 1000 grasping the pump member 60, and positioned to manipulate the switch 1000 including toggle 1001 (having an alternate position 1001).

FIGS. 6A, 6B, and 6C are related drawings showing the use of a heel lock concept.

FIG. 6A shows a foot support pad 33 including a heel lock apparatus 90 including grasping members 91 and an adjustment member 92. FIG. 6B shows the heel lock apparatus 90 attached to the sole of the shoe of a wearer. This shoe might be a conventional shoe, or may be a special shoe. FIG. 6C shows an alternative heel lock apparatus 90A which grasps the entire shoe of the wearer.

FIG. 7 is a hydraulic system layout which illustrates a “Version One” layout.

FIG. 8 is a hydraulic system layout which illustrates a “Version Two” layout.

FIG. 9 a hydraulic system layout which illustrates a “Version Three” layout.

FIG. 10 is a hydraulic system layout which illustrates a “Version Four” layout.

FIG. 11 is an illustrative view of an illustrative accordion action. Theta (1) and (2) show different angles at which the foot plate can be fixed.

FIG. 12 shows an alternate heel lock apparatus 190, which includes a heel lock feature by use of an ankle cuff 192 which is attached to the foot pad 33 by adjustable straps such as generally shown as 194. By adjusting the straps the heel 2001 of the foot 2000 of the leg 2002 of the user can be “locked” relative to the foot pad 33. Another use of the straps 194 is to provide adjustable connections between points 195 and 196, so as to also function to secure the position of the foot pad 33 relative to the sliding mount 31 of the foot support carriage assembly. Note the side and rear straps are shown; a “front” strap would also likely be used in order to provide adequate locking of the foot pad 33 as desired.

FIG. 13 shows an alternative embodiment of the invention, which shall be hereinafter referenced as compliance apparatus 200 (a.k.a. “Compliance Version One apparatus 200”), which includes a section 210 which includes elements which are similar to the previously described embodiment, but also includes some additional elements, notably the compliance-related data accumulator assembly 250.

FIG. 14 shows a more detailed perspective view of the data accumulator assembly 250, which includes a data accumulator readout panel 255, which in one embodiment provides a numerical readout corresponding to the amount of time (in minutes, hours, etc.) that the patient has exerted a preselected threshold amount of force against the foot support carriage assembly 30.

FIG. 15 shows an even more detailed perspective view of the data accumulator assembly 250, albeit with a cover panel removed for view of its contents. This view of the data accumulator assembly 250 shows a pressure switch 252, a data accumulator timer 254, and a data accumulator readout panel 255. Also shown is a portion of the high side portion of the fluid lines 230, with a fluid “T” member 238 providing a connection of high side portion of the fluid lines 230 to the pressure switch 252.

FIG. 16 shows a front elevational view of that shown in FIG. 15. This view of the data accumulator assembly 250 shows a pressure switch 252, connecting wires 253, a data accumulator timer 254, and a data accumulator readout panel 255. Also shown is a portion of the high side portion of the fluid lines 230, with a fluid “T” member 238 providing a connection of high side portion of the fluid lines 230 to the pressure switch 252.

FIG. 17 is illustrative view of a data accumulator assembly 350 for use with Version 2 of one embodiment of the present invention, which includes a readout panel 351, a fluid pressure transducer 352, an antenna 360, and a direct download port 362. The data accumulator assembly 350 receives inputs from a carriage location sensor 356, a seat sensor 358.

FIG. 18 shows a block diagram including the central storage device 400, an administrative user 402, various exemplary medical professionals 410, and exemplary Compliance Version Two apparatuses 300.

FIG. 19 is an illustrative view showing the relative torque and angular displacement encountered by two different legs, shorter leg L1 (shown in solid line) and longer leg L2 (shown in dotted line) for a given position of the foot support carriage assembly 30 and chair 5.

FIG. 20 shows the leg of one user (a.k.a., patient) at two different carriage positions P1 and P2, the first position P1 being further extended than the other (P2).

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

General Operation

Generally described, the present invention is provided by an overall orthotic apparatus 10, which includes the following components:
a frame 20;
a foot support carriage assembly 30;
a linear force output assembly 40;
a fluid pump and diversion assembly 50;
a manual pump member 60; and
a spring return apparatus 70.

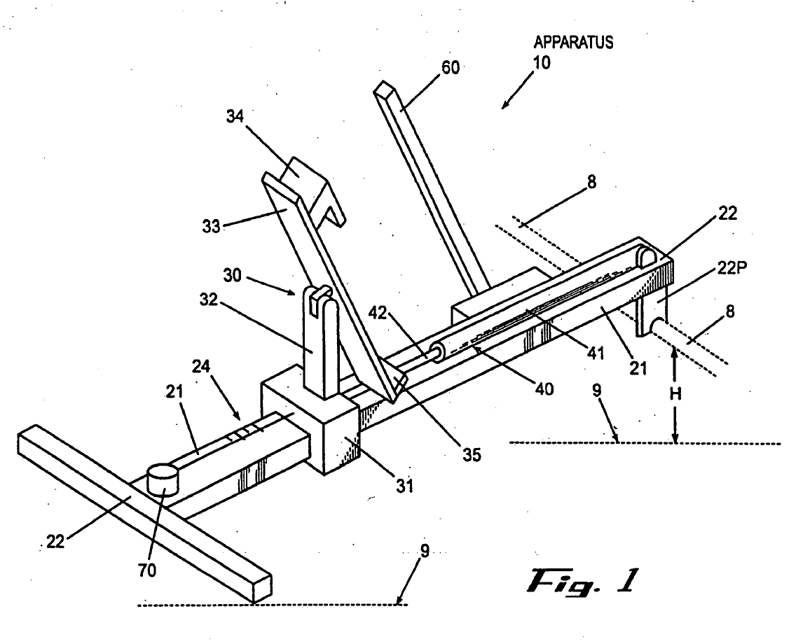

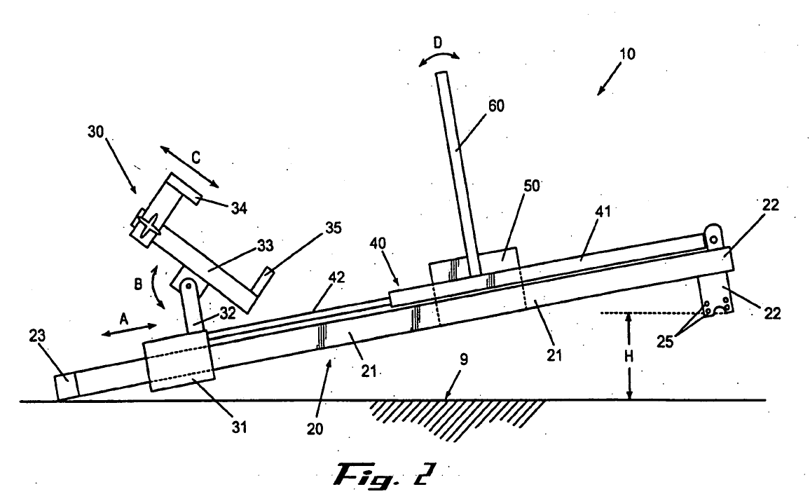

A chair 5 is used in conjunction with apparatus 10 according to the present invention. As discussed in further detail below, the chair includes a cross rail which is configured to support the head 22 of the frame 20 of the overall apparatus 10 such that the apparatus is in a relatively inclined position as shown in FIGS. 1 and 2, and such that the apparatus 10 is detachably attached to the chair.

Detailed Discussion

More details are now provided.

Elements List

The overall list of elements discussed herein includes the following:
5 Chair
6 Chair leg
7 Chair seat
8 Chair cross member
9 Supporting surface
10 Overall apparatus
20 frame
21 spine
22 head
22P slotted plates
23 foot
24 linear travel markings
25 slotted plate holes
30 foot support carriage assembly
31 sliding mount
32 pedestal
33 foot support pad (a.k.a. foot support plate)
34 toe capture member
35 heel stop
40 linear force output assembly
41 piston shell (a.k.a. “cylinder”)
42 piston rod
50 fluid pump and diversion assembly
60 manual pump member
70 Spring return apparatus
90 Heel lock apparatus
100 Switch
101 Switch toggle
190 Alternative Heel Lock
192 Ankle apparatus cuff
194 Straps
195 Point
196 Point
1000 user
1001 user right leg
1002 user left leg
1003 user right hand
1004 user left hand
1005 user right foot
1006 user left foot

The Overall Apparatus 10

As noted above, the overall apparatus 10 includes a frame 20, a foot support carriage assembly 30, a linear force output assembly 40, a fluid pump and diversion assembly 50, a manual pump member 60, and a spring return apparatus 70.

The Frame 20

Referring generally to FIGS. 1-4, The frame 20 includes a generally elongate spine 21, a head 22, a foot 23, and linear travel markings 24. The frame 20 is configured to remain relatively stationary when the apparatus is being used. The linear travel markings 24 are configured to allow a user to determine the extent to which the foot support carriage assembly 30 has moved relative to the spine 21 of the frame 20.

The spine 21 is generally elongate, and has the head 22 fixed at one end and the foot 23 fixed at the other end. When installed, the spine 21 of the frame 20 is slightly inclined upwardly from the foot to the head ends.

The head 22 includes a pair of slotted plates 22P, each providing a slot, with each slot configured to accept a cross member (or bar) 8 of a conventional folding chair such as shown in FIG. 3. As may be seen, the cross member 8 extends between the two rear legs 6 of the chair and is substantially horizontal and transverse to the spine 21 of the overall apparatus when the overall apparatus 10 is in use. As will also be seen, this allows for a significant amount of opposing forces to be applied to the back of the chair and the foot support pad of the apparatus. Furthermore there can be holes 25 (see FIG. 3) in cross member 8 the head member of the apparatus 10 which allow for plastic or other suitable tie wraps (not shown) to be placed in such a manner as to lock the cross member 8 of chair 5 to the apparatus.

The foot 23 of the frame is likewise generally elongate, and extends relatively transverse to the longitudinal axis of the elongate spine 21. The foot 23 is configured to provide stability at the point at which the frame 20 contacts a typical supporting surface. Therefore it may be seen that the frame 20 is supported at two general locations, the head 22 (resting on the chair) and the foot 23 (resting on the supporting floor surface).

Foot Support Carriage Assembly 30

The foot support carriage assembly 30 is configured to slide along a track defined by and relative to the frame 20. Particularly, the linear force output assembly is configured to slide along a relatively straight axis, which is parallel to the longitudinal axis on the elongate spine 21 of the frame 20. The foot support carriage assembly 30 includes a sliding mount 31, a pedestal 32, a foot support pad 33, a toe capture member 34, and a heel stop 35.

The sliding mount 31 of the foot support assembly 30 is configured to slide relative to the spine 21 of the frame 20 by the use of nylon bearings or other suitable means known in the art.

The pedestal 32 of the foot support assembly 30 extends substantially vertically upwardly from the sliding mount 31, and is relatively rigidly mounted relative to the sliding mount 31.

The foot support pad 33 is pivotably attached relative to the upper end of the substantially vertical pedestal 32, such that as a user flexes his/her ankle, the foot support pad may be pivoted about an axis which is relatively transverse to the longitudinal axis of the spine 21. In the preferred embodiment, the foot support pad is pivotable about a substantially horizontal axis which is transverse to the longitudinal axis of the spine 21.

As may be understood, the foot support pad 33 is configured to support and be in contact with the sole of the shoe of a user, although of course a bare or stocking foot may be used as well.

Toe capture member 34 is configured to be releasably but slidably mounted relative to along a portion of the length of the support pad 33. The heel stop 35 is relatively rigidly mounted relative to the foot support pad 33. The toe capture member 34 is configured to combine with the heel stop 35 to allow the foot support carriage assembly 30 to capture the foot of a user.

It may be understood that, by tightening and loosening suitable clamping members, the location of the toe capture member 34 may be adjusted along the length of the somewhat elongate foot support pad 33. Therefore, it may be further understood that this foot support pad system may be adjusted depending on the size of the user's foot as needed in order to engage the wear's foot relative to the carriage assembly 30.

When in operation, the foot support pad 33, toe capture member 35 and heel stop 35 combine to pivot together as needed. When the invention is used in “knee flex mode” (as a “Knee Flexionater™”) the foot support pad is allowed to pivot relative to the linear force output assembly. This allows the ankle to become more plantar flexed as the knee is flexed (the foot support pad moves toward the chair). When the invention is used in “ankle flex mode (as an “Ankle Flexionater™”) the foot support pad is fixed in a particular angle with respect to the linear force output assembly. The angle is set dependent upon the patient and his/her situation. With the foot support pad fixed (unable to pivot) the ankle joint is forced into dorsiflexion as the knee is flexed (the foot support pad moves toward the chair). This process is best visualized by the angles of an accordion as shown in FIG. 11. When the accordion's bellows are stretched fully (as in Version “A”) out the angle between each bellow is wide but when the accordion's bellows are squeezed tightly together (as in Version “B”) the angle between each bellow is very acute. The same occurs with the hip, knee and ankle. As the foot pad is moved toward the chair the hip and the knee are necessarily flexed, and, as follows, the ankle must flex also, if the foot pad angle is fixed.

Linear Force Output Assembly 40

The linear force output assembly 40 includes a piston shell 41 and a piston rod 42. In practice, the linear force output assembly 40 is a hydraulic cylinder.

The linear force output assembly 40 is attached with one end to the foot support carriage assembly 30 and with the other end to the frame 20 of the apparatus 10. The linear force output assembly 40 is configured to provide opposing (pulling) forces at each of its ends, and opposing pushing forces in certain variations. In the configuration shown, this allows a force to be provided on the foot support carriage assembly 30, which causes the foot support carriage assembly 30 to be moved along a straight axis in a reciprocating manner along the spine of the frame 20. As will be discussed in later detail, the linear force output assembly 40 is configured to be moved upon the movement of a manual pump member 60 by the user.

Fluid Pump and Diversion Assembly 50

The fluid pump and diversion assembly 50 is comprised of a plurality of hoses, valves, etc., which provide a pumping action to a fluid as needed, as well as providing various diversions of the flow of the fluid within the fluid pump and diversion assembly.

The manner in which the fluid pump and diversion assembly 50 can operate includes several versions, all of which include the use of a hand pump, a reservoir tank, a main piston (e.g., 40) and a switch (e.g., switch 100 having a toggle element 101).

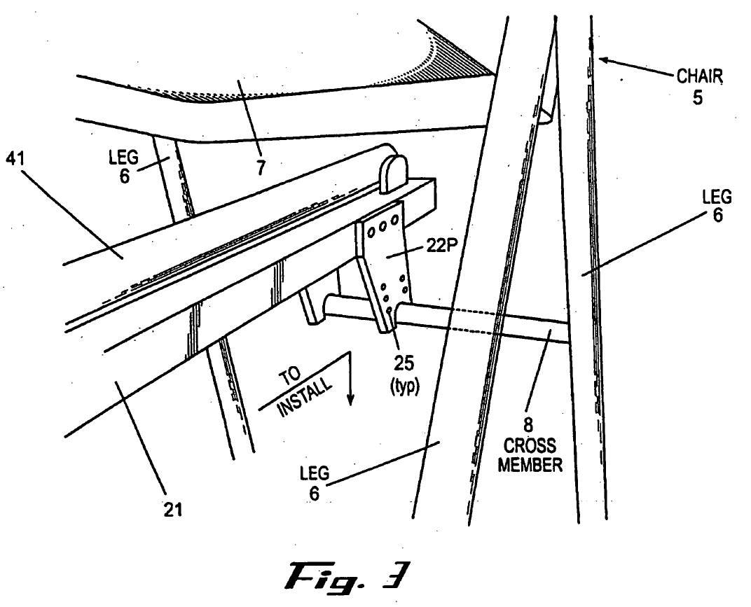

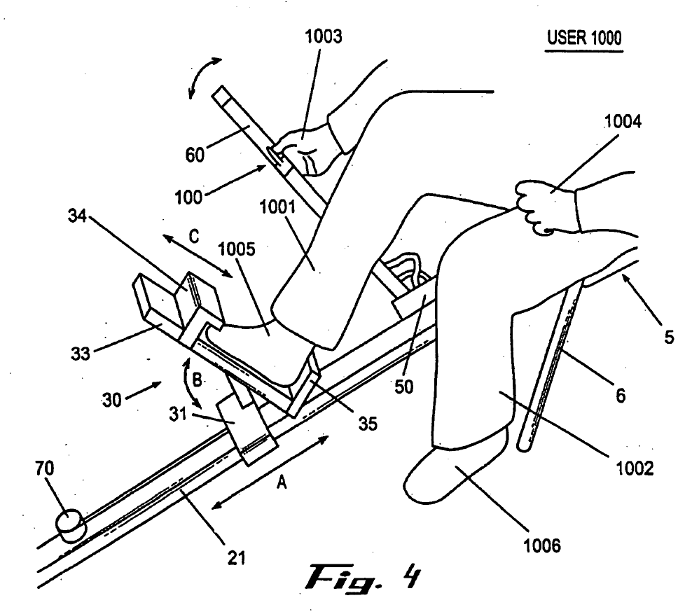

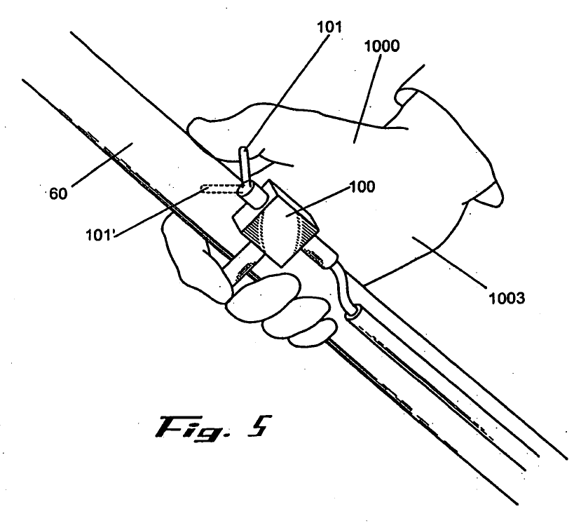

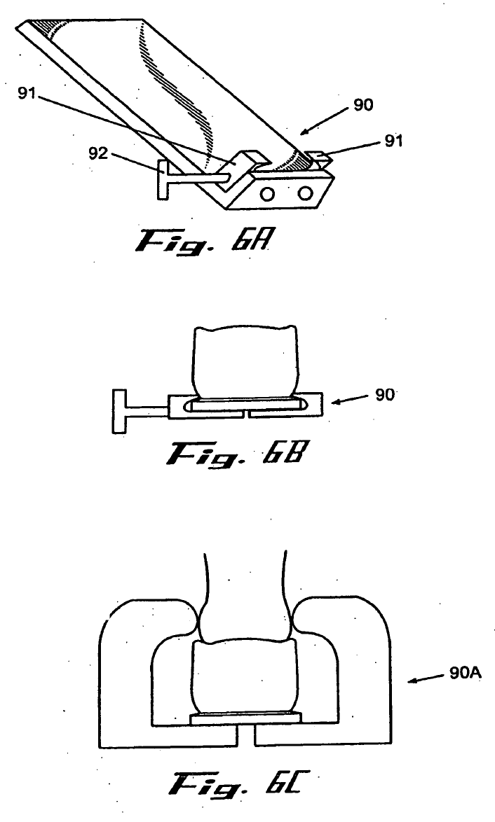

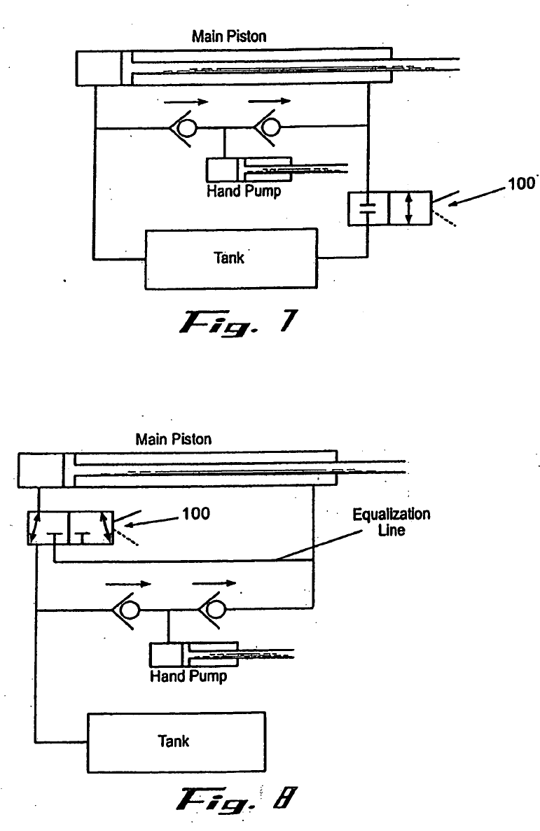

Version One—Version One is a First Configuration, Shown in FIG. 7.

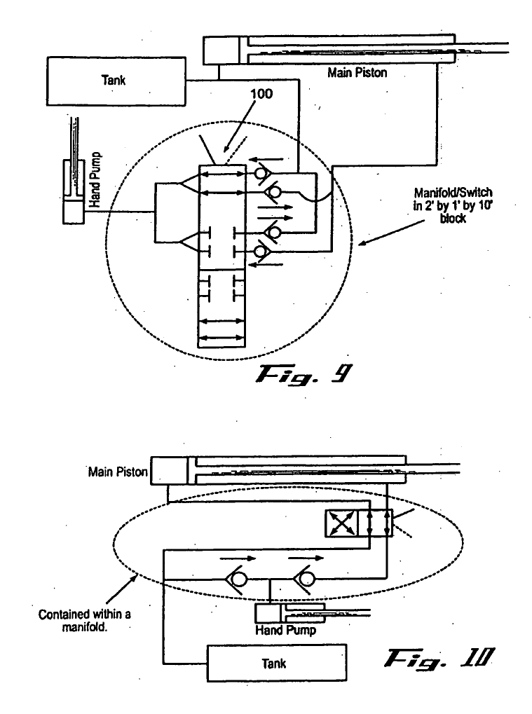

Version Two is shown in FIG. 8—In Version Two, no spring return is used. The hand pump is used to pressurize the right side of the cylinder, thus moving the piston rod towards the left as the figure is viewed (thus further bending the knee) when the switch is in the “pump” position (toggle switch shown in solid line). This dumps fluid in the left portion of the cylinder in a line leading towards the tank. When the switch is in the “release” position (toggle switch shown in dotted line), pressure within the main piston cylinder is equalized between both compartments. As more fluid is pumped into the system the differential between the area of the side of the internal piston without the pump rod the area with the pump rod causes the rod to extend eliminating the need for a spring return.

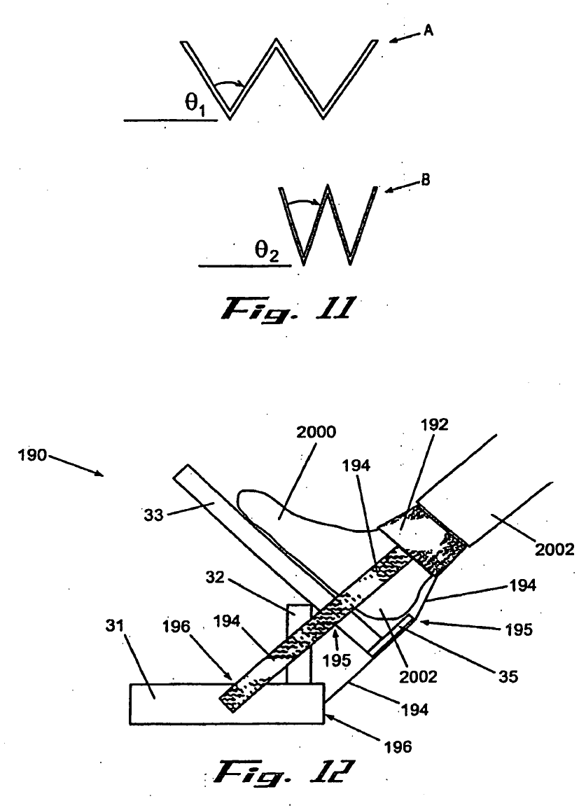

Version Three is shown in FIG. 9—In Version Three, the use of the switch allows the user to pump the knee into extension, obviating the need for a spring return.

Version Four is shown in FIG. 10—Version Four likewise includes the use of the switch which allows the user to pump the knee into extension, obviating the need for a spring return.

Manual Pump Member 60

The manual pump member 60 is essentially a hand-held lever which extends relatively upwardly from the fluid pump and diversion assembly 50. In practice, this member 60 may be pivoted by a point adjacent to somewhat above the spine 21 of the frame 20, but it may also be allowed to pivot “side-to-side” as needed in order to allow some leeway for the user to allow use of the manual pump member by either the right or left hand as needed.

Spring Return Apparatus 70

The spring return apparatus 70 provides a spring return feature by use of a string wound on a reel, with the reel being spring loaded by use of a torsion or other suitable spring. The frame of the spring return apparatus 70 is mounted relative to the frame of the overall apparatus 10. In the preferred embodiment the spring return apparatus 70 provides a substantially constant five-pound force on the foot support carriage assembly 30 relative to the frame of the apparatus.

Use in Conjunction with Chair

One important feature of the invention is its use in conjunction with a chair such as 5.

As may be understood, this allows for separation of the elements 5, 10, to allow for separate and easy storage. Reference is made to FIG. 3 to show the manner and direction of installation, which is simple yet very effective. Disinstallation is the opposite of installation.

Operation

In order to operate the device, the following steps are typically used.

The user situates the apparatus 10 relative to the chair 5 as shown in FIG. 1. The user is then seated in the folding chair 5, such that the sole of the user's foot is situated atop the foot support pad 33 of the foot support assembly 30. The user then manipulates and secures the toe capture member 34 such that the toe capture member combines to capture the foot of the user when used with the heel stop 35.

The user typically will place the foot atop the foot support carriage assembly 30 when the user's foot is relatively extended. The switch 100 according to the present invention is then positioned to its “closed” position by manipulation of the toggle element 101.

The manual pump member 60 is then pivoted in a reciprocating manner, to provide a “pumping” action to a hydraulic pump so that fluid is moved from the pump under pressure to the linear force output assembly 40, causing the linear force output assembly to stroke in its out erection.

Such stroking causes the fluid support assembly to move towards the chair, such that the user's leg tends to be bent. As may be understood, eventually the user may encounter some pain or discomfort as the leg is bent. When the user can no longer sustain the discomfort, the user is then allowed to “flip the switch” (the toggle 101) of the switch 100, which causes relief on the bent leg.

Referring to FIGS. 2 and 4, movement “A” is linear relative movement between elements 31 and 21. Movement “B” is pivotal movement between elements 33 and 31. This can be fixed or free. Movement “C” is linear sliding movement between elements 34 and 33, which is occasional as needed for adjustment. Movement “D” is relative pivoting movement of element 60 relative to, for example, element 21.

The Heel Lock Feature

The Heel Lock feature of the present invention provides a locking feature to releasably attach the heel of the user relative to the foot support plate 33. This is advantageous in that during the accordion effect, which has been previously described, the heel of the foot in the foot pad will have a strong dorsiflexion moment applied across the ankle during the process. Since dorsiflexion is the intended motion to obtain during the treatment process due to the lack of same, the heel will have a force pushing it out of the foot pad. This heel lifting force is counteracted by a heel lock feature. This heel lock feature can be composed of a clamp on a shoe sole, clamp on a heel cup of a shoe or a harness type of strap around the ankle holding the heel down to the foot pad.

The Toe Capture Feature

As noted above, the toe capture member 34 is configured to be releasably but slidably mounted relative to along a portion of the length of the support pad 33. The heel stop 35 is relatively rigidly mounted relative to the foot support pad 33. The toe capture member 34 is configured to combine with the heel stop 35 to allow the foot support carriage assembly 30 to capture the foot of a user. This provides for an advantageous Toe Capture feature which provides improved operating characteristics. As the footpad is moved towards the chair the knee is flexed. During this process the footpad, if allowed to pivot freely, moves into a position parallel with the linear force output assembly. When the footpad is parallel to the application of force used to move the footpad toward the chair, the foot will slide out of the footpad negating the applied flexion moment at the knee without the use of the toe capture feature. With the toe capture feature the pressure on the foot is maintained.

The toe capture feature is only needed when the device is used as a Knee Flexionater.

The Ankle Lock Feature

As noted above, the foot support pad 33 is pivotably attached relative to the upper end of the substantially vertical pedestal 32. The Ankle Lock feature of the present invention provide a locking feature to releasably fix the position of the foot support pad relative to the pedestal 32 as well as the sliding mount 31 This is advantageous in that it is essential to the accordion effect. The foot support pad must be angularly fixed with respect to the slide mount 31 during operation of the device in order for the accordion effect to exert a dorsiflexion moment at the ankle. This ankle lock feature must be allowed to fix this angle at any angle in accordance with the needs of the patient.

Note again that the Heel Lock and Ankle Lock features must be used together when the device is used as an Ankle Flexionater.

For use of the Ankle Flexionater™ the clinician sets up the device by extending the leg and fixing the angle of the foot plate at a comfortable position for the patient. The Heel Lock™ is then clamped around the sole or last of the heel of the patient's shoe or a harness as discussed later is attached to the ankle and attached to the foot pad. The toe clamp is not necessary for this process and is moved out of the way. This then allows the patient to slide his shoe down into the adjusted Heel Lock™ clamp during every session to keep the heel of the shoe from raising off of the foot plate during the stretching process. The patient must use a tie up shoe in order to facilitate the use of the clamp system. In cases where the clamp is insufficient a Velcro™, buckle strap, or other suitable system could be used around the ankle as a harness to hold the heel down to the foot plate during the stretching process. The manual pump member is then pivoted in a reciprocating manner similar to the use of the Knee Flexionater™ causing the knee to flex and, due to the biomechanics of the lower extremity, the ankle will be also forced into dorsiflexion.

Alternate Heel Lock and Ankle Lock Configurations

FIG. 12 shows an alternate heel lock apparatus 190, which includes a heel lock feature by use of an ankle cuff 192 which is attached to the foot pad 33 by adjustable straps such as generally shown as 194. By adjusting the straps, the heel 2001 of the foot 2000 of the user can be “locked” relative to the foot pad 33. Another use of the straps 194 is to provide adjustable connections between points 195 and 196, so as to also function to secure the position of the foot pad 33 relative to the sliding mount 31 of the foot support carriage assembly, providing the “Ankle Lock” function as well.

Note the side and rear straps are shown; a “front” strap would also likely be used in order to provide adequate locking of the foot pad 33 as desired.

Furthermore, the ankle lock device could also include a flat circular plate having holes therein, that could be rotated and pinned in position (through the holes) to 33 or 32 in FIG. 2. As an example, the plate could be attached to member 32 with a long pin to attach the plate relative to element 32. It could be a gear mechanism, or toothed clamp. One could imagine a dozen ways to allow the motion between the member 33 and member 32 to be restricted occasionally and released when necessary.

Treatment Protocol Used with Original Device 10

Under one current treatment protocol, patients (a.k.a., “users”) are instructed to conduct multiple separate treatment sessions, such as three sessions a day, with each session including a certain amount of stretch time (e.g., twenty minutes) of “stretch” time, which does not include intermediate “relax” time. In this example, this provides one full hour of what could be referenced as “stretch” and “relax” therapy. “Stretch” (or “contracting”) of the knee should be understood in the context of this application as bending of the knee under pressure while experiencing torque on the knee, “relaxing” of the knee should be understood as relaxing the knee with the absence of torque, or at least less torque.

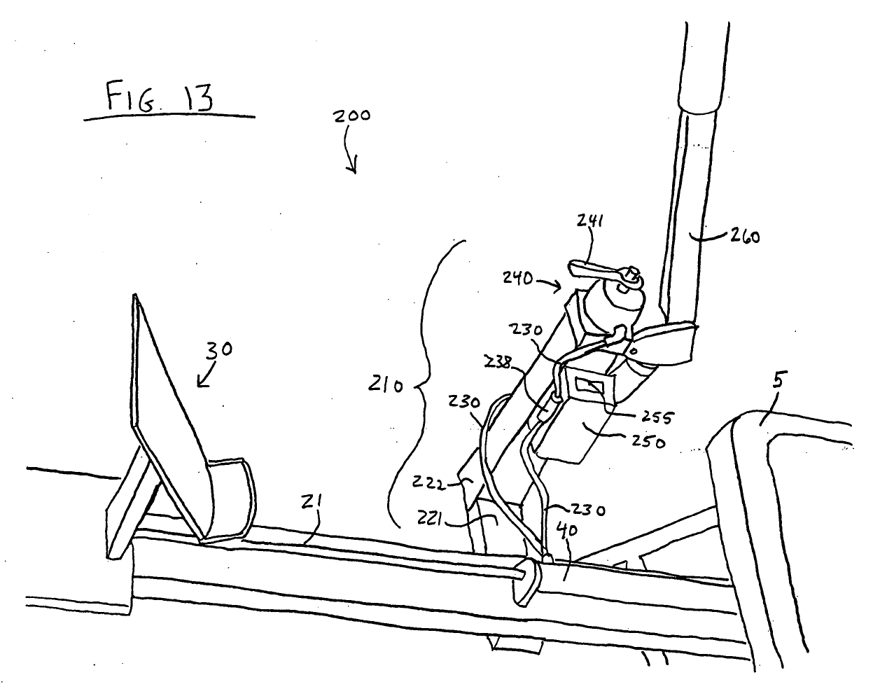

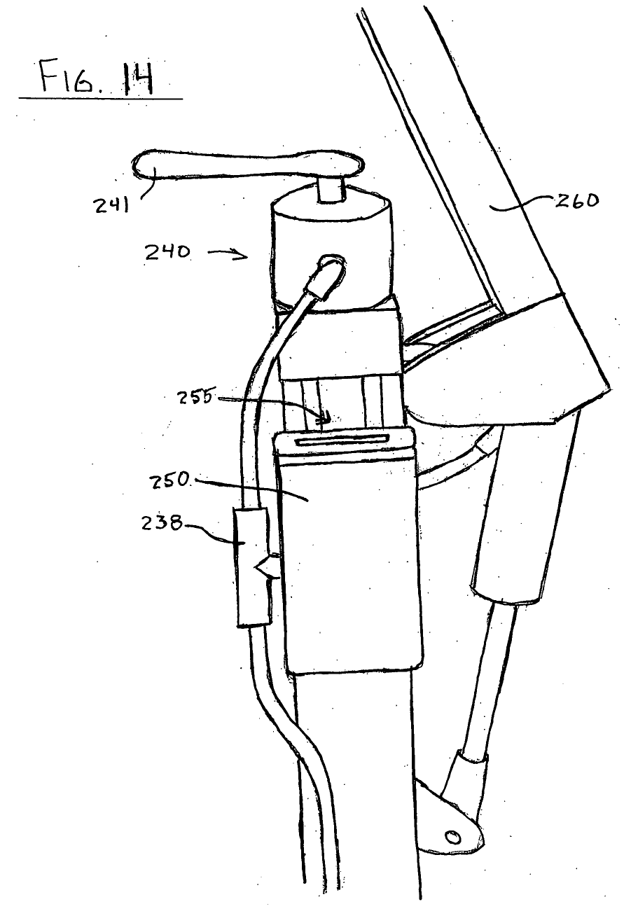

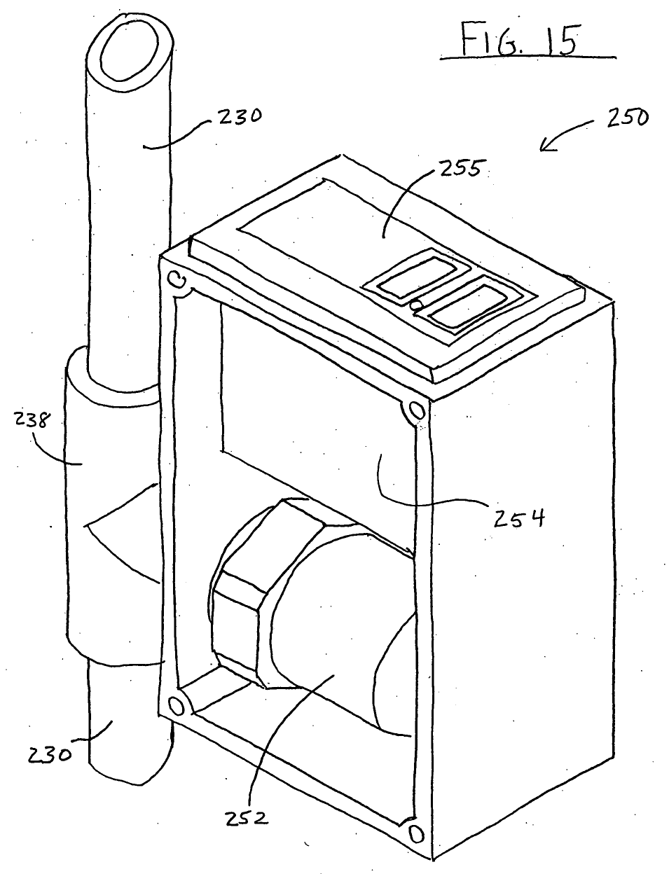

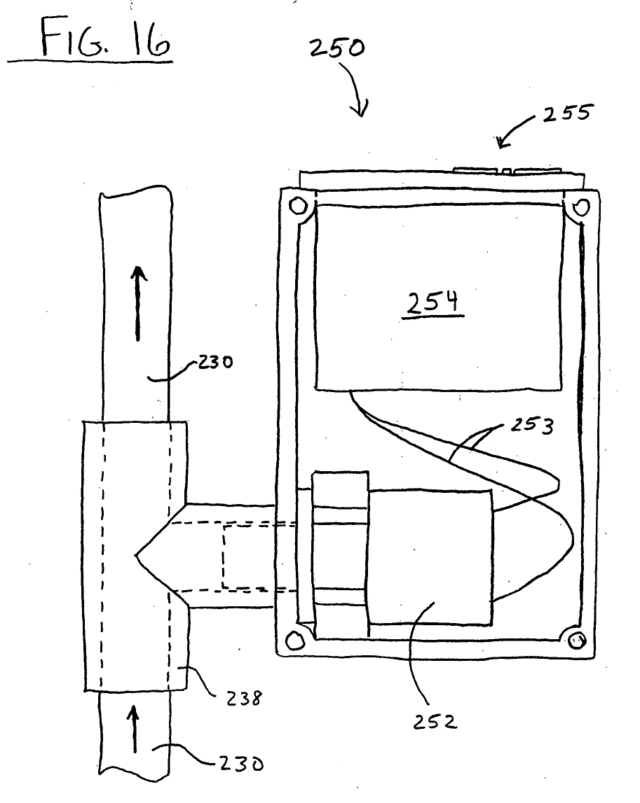

At the beginning of a session, a patient is asked to use the apparatus 10 such that the knee is stretched (“bent”) as much as possible, within pain limitations, and to hold this position as long as possible. If the patient experiences excessive discomfort, the load on the knee could be released somewhat by manipulating the relevant pressure relief devices (100 in FIG. 4, 241 in FIG. 13) such that just enough load is reduced so as to allow just enough discomfort to be lessened, while still stretching the knee as much as possible. The point is to try to keep as much torque on the knee for as long as possible, for therapeutic reasons.

The patient is asked to keep the knee so stretched for a certain time period, for example ten minutes. The patient can then completely release the load (by manipulating the relevant pressure relief devices and allowing the leg to extend) and rest for a similar time period, in this case another ten minutes. Resting can include remaining in the device, or walking around and thus manuplating the joint. After the rest period, the patient is asked to repeat the previous stretching step, keeping the knee in as much stretch as possible for another period of time, such as another ten minutes. This would in one example complete one session with twenty minutes of stretch time. Under the protocol requiring one hour of stretch time, this would correspondingly require three sessions per day, which could be done with the first in the morning, second at midday, and the third in the evening.

For some patients, especially in the initial stages of recovery, ten minutes is a relatively long time and can become painful. If this is the case, the patient could be instructed to make the stretching and relaxing periods shorter but still relatively equal (five minutes on, five minutes off, five minutes on, etc.) as long as the total stretch time totals twenty minutes.

Challenges

As may be understood, there can be challenges in following and substantiating such protocols. For example, patients often do not keep good records of their use of the device. Often it is difficult for a health care professional (such as a nurse, physical therapist, doctor, or health care insurance provider) to know if a protocol is being complied with—a patient instructed to use the device for one hour could be using the device for only twenty minutes, or two hours. Assuming the one hour protocol is desired, such variations could impede or even reverse patient progress.

Thus there is obviously an interest in understanding how such devices are being used for the particular patient (so the particular patient can be appropriately treated). However there is also a broader, longer term interest in understanding how the broader patient population responds to a given protocol; if trends can be spotted which indicate that certain aspects of the protocols work better than others, future patients can benefit from modified and improved protocols.

In light of these challenges, various “compliance” embodiments are contemplated under the present invention.

Compliance Version 1

Description of Compliance Apparatus 200 (a.k.a. Compliance Version One apparatus 200)

Reference is now made to FIG. 13, which shows an alternative embodiment of the invention, which shall be hereinafter referenced as compliance apparatus 200 (a.k.a. Compliance Version One apparatus 200), which includes a section 210 which includes elements which are the same as in previously described embodiment 10 (e.g., the chair 5, the spine 21, the foot support carriage assembly 30, for example), similar in operation to the previously described embodiment 10 (e.g., the releaseibleed valve 240 having an operating handle 241), but also includes some additional elements, notably the compliance—related data accumulator assembly 250. As described in further detail below, this data accumulator assembly accumulates data indicative of the use of the overall apparatus 200, such as when a given patient is instructed to conduct multiple separate treatment sessions, as an example three sessions a day, with each session including a certain amount of “stretch” time (as an example twenty minutes), which does not include intermediate “relax” time. In this example, this provides one full hour of “stretch” and “relax” therapy. Again, “stretch” of the knee should be understood as bending of the knee under pressure while experiencing torque on the knee, “relaxing” of the knee should be understood as relaxing the knee with the absence of torque.

Element List

Here follows a list of elements within the first compliance apparatus embodiment.
200 Compliance apparatus (a.k.a., Compliance Version One apparatus)
210 Section
221 Frame member
222 Frame member
230 Fluid lines
238 Fluid line “T”
240 Pressure release/bleed valve
   241 Operating handle
250 Data Accumulator Assembly
   252 Pressure switch
   253 Connecting wires
   254 Data Accumulator Timer
   255 Data Accumulator Readout Panel

Frame Members

As will be seen, the section 210 includes frame members 221 and 222, which are attached rigidly relative to the spine 21. These frame members 221 and 222 provide support for the manual pump member 260, so the manual pump member 260 can be pivoted relatively thereto in manner similar to that described above so as to provide pressurization within the fluid lines 230 so as to cause the linear force output assembly 50 to operate as described before.

The frame members 221 and 222 also provide support for pressure release/bleed valve 240 (having an operating handle 241) which operates in a manner similar to the pressure switch 100 and toggle 101, respectively, of system 10 of FIG. 4.

The frame members 221 and 222 also provide support for data accumulator assembly 250, described below.

Data Accumulator Assembly 250

The data accumulator assembly 250 is configured to provide a visually perceptible readout and/or machine readable data signal corresponding to use of the device.

In one preferred embodiment, this readout and/or machine readable data signal corresponds to the total amount of time (since last reset) that a patient has exerted a threshold amount of force against the foot support carriage assembly 30. This is accomplished by operable linking data accumulator assembly 250 into the fluid lines 230 at a point at which the accumulator assembly 250 can accumulate data relating to the pressure of the fluid within a fluid line which provides pressure causing the force output assembly to extend (hereinafter the “high line”). This allows for data to be accumulated relating to the force being exerted by the device 200 on the leg of the patient, whether or not the foot support carriage assembly 30 is moving or not. When combined with the use of a timer, this allows the data accumulator assembly 250 with the capability of logging how long the pressure in the high line has been above a given threshold, which can be selected as desired, typically upon setup of the device.

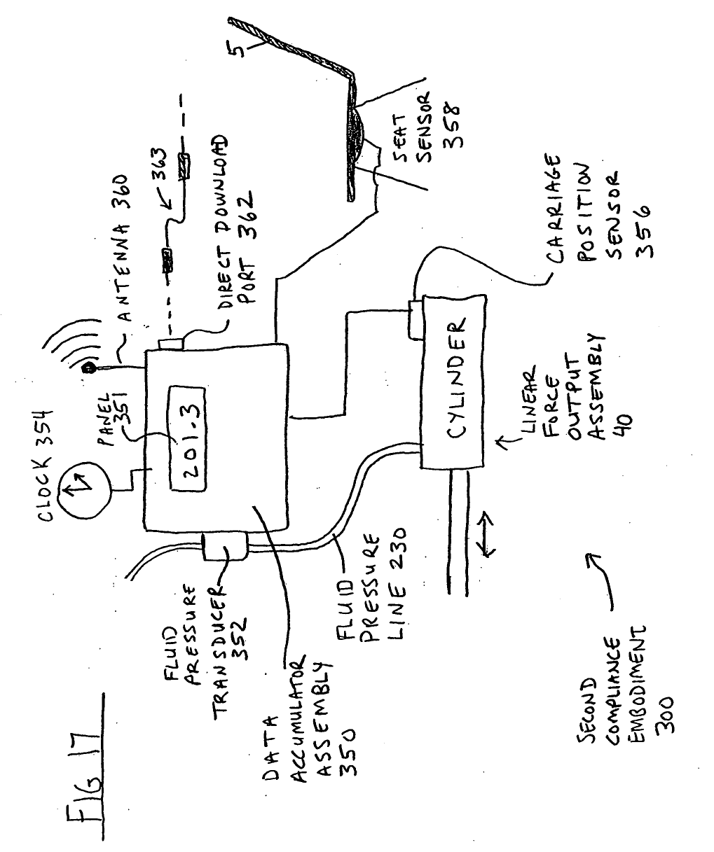

In one preferred embodiment, this includes the use of a data accumulator assembly 250 such as shown in FIGS. 15 and 16, showing the data accumulator assembly 250 including a pressure switch 252, connecting wires 253, a data accumulator timer 254, and a data accumulator readout panel 255. Also shown is a portion of the high side portion of the fluid lines 230, with a fluid “T” member 238 providing a connection of high side portion of the fluid lines 230 to the pressure switch 252.

It should be understood that the data accumulation device 250 could be used as an optional accessory on the original device, added by technical personnel providing the devices as needed to certain patients as necessary.

Pressure Switch 252

The pressure switch 252 is a switch that activates depending on the pressure it senses, which in this case is the pressure within the high side portion of the fluid lines 230. Under one embodiment of the invention, this is a simple closed vs. open switch, and closes when pressure within is past a certain threshold, which in one example can be 50 PSI. However it should be understood that this threshold could be a varied to different PSI settings depending on needs and/or conditions. The selection of the PSI setting is, however, selected in a manner so as to give a reasonable understanding of when the device could be assumed to be in “stretch” mode.

Connecting Wires 253

Connecting wires provide a signal path between the pressure switch 252 and the timer 254.

Data Accumulator Timer 254

The data accumulator timer 254 in the embodiment shown is a timer that logs the lapsed time that the pressure switch is closed (due to the pressure within the high side portion of the fluid lines 230 reaching a predetermined level). As may be understood, this lapsed time is not necessarily going to be a one single time period that that the pressure switch is closed, but an accumulation of a series of separate time periods, between which the switch was open.

Data Accumulator Readout Panel 255

The data accumulator readout panel 255 provides a visually perceptible indication of the elapsed time (since last reset) that the pressure switch has been closed. Thus it may be understood that the data accumulator readout panel 255 provides a visually perceptible indication of the elapsed time (since last reset) that device could be assumed to be in “stretch” mode.

Data Readings and Conversions

Readings from the data accumulator readout panel 255 of the data accumulator assembly 250 could be read by the patent, or by a health care professional, or by those providing technical service to the devices. Although the version shown in the drawings only shows a accumulator assembly 250 having a readout panel 255 for providing data output (obtained by reading the panel 255) in an alternative configuration a wireless modem could be used if so desired assuming additional equipment is put into place.

Treatment Protocol Used with Version 1

The treatment protocol used for Version 1 can be generally similar to that described above; a given patient is instructed to conduct multiple separate treatment sessions, as an example three sessions a day, with each session including a certain amount of “stretch” time (as an example twenty minutes), which does not include intermediate “relax” time. In this example, this provides one full hour of “stretch” and “relax” therapy.

However, this protocol of use by the patient could also include the use of the data accumulator readout panel 255, which is configured to be viewable by the patient while using the device. As noted above this panel provides a visually perceptible indication of the elapsed time (since last reset) that the pressure switch has been closed; the patient could use the data accumulator readout panel 255 to determine when the appropriate amount of “stretch” time has been accomplished, be it for the particular session, the three-session day, for the week, etc. This could even be a part of a prescribed order by a physician (patients are told to try to log at least 1 hour a day, etc.).

Data Output and Analysis of Data from Version 1

As noted above, a patient could use the data shown by the data accumulator readout panel 255 to determine when the appropriate amount of “stretch” time has been accomplished, be it for the particular session, the three-session day, for the week, etc.

However, others could also use such data; a health care professional such as a physical therapist, nurse, doctor, insurance provider, or the like, could also use the data accumulator readout panel 255 to better determine how much the patient has used the device since patient last visited a health care professional or was visited by same. The information gleaned from the use the data accumulator readout panel 255 could assist the health care professional in making a decision whether or not to continue use of the device with the patient, and/or to modify the therapy schedule.

Data from the data accumulator readout panel 255 could also be used to better understand the amount of time the unit has been in service since the readout panel has been reset. This could be used in determining if the overall apparatus may be due for periodic service.

Finally, a health care research professional could use the data along with other recorded data regarding client success to research and develop a new protocol as needed.

Advantages of Version 1

It has been found that some patients use the device similar to an exercise device, in that they include multiple, relatively rapid, repetitions without keeping the knee stretched for an appreciable amount of time, which is not recommended. The provision of the data accumulator assembly 250 and its readout panel 255 (which shows the amount of time the knee is undergoing some kind of torque and which could only be a small part of an “exercise”—type action) could encourage the patient to curtail “exercise”—type action, and instead use the device more as prescribed.

Compliance Version 2

Description of Compliance Version 2 (a.k.a. Second Compliance Apparatus)

Reference is now made to FIG. 17, which shows a Compliance Version Two apparatus 300 according to one embodiment of the invention, which includes elements which are similar to the previously described embodiments 10 and 200 (such as a linear output assembly 40, a chair 5, a frame (not shown), and a foot support carriage assembly (not shown)). However, the Compliance Version Two apparatus 300 includes additional features shown in FIG. 17 which accumulate data indicative of the use of the apparatus 300. This use can be such as that described above with respect to apparatuses 10 and 200, such as when a given patient is instructed to conduct multiple separate treatment sessions, as an example three sessions a day, with each session including a certain amount of “stretch” time (as an example twenty minutes), which does not include intermediate “relax” time. In this example, this provides one full hour of “stretch” and “relax” therapy. Again, “stretch” of the knee should be understood as bending of the knee under pressure while experiencing torque on the knee, “relaxing” of the knee should be understood as relaxing the knee with the absence of torque.

Element List

Here follows a list of elements within the second compliance embodiment apparatus.
300 Compliance Version Two Apparatus
350 Data Accumulator Assembly
351 Readout Panel
352 Fluid Pressure Transducer
354 Clock
356 Carriage Location Sensor
358 Seat Sensor
360 Antenna
362 Direct Download Port
400 Central Data Storage Device
402 Administrative User
410 Exemplary Medical Professionals

Data Accumulator Assembly 350

The data accumulator assembly 350 of FIG. 17 is configured to accumulate data for subsequent transmission to an external source, and could optionally provide an onboard reading for use by the patient or other personnel via readout panel 351.

It should be understood that the data accumulation device 350 could be used as an optional accessory on the original device, added by technical personnel providing the devices as needed to certain patients as necessary.

Data Accumulator Assembly 350 Inputs

The data accumulator assembly 350 of FIG. 17 includes multiple potential inputs and outputs. The following are potential inputs.
352 Fluid Pressure Transducer
354 Clock
356 Carriage Location Sensor
358 Seat Sensor

Fluid Pressure Transducer 352

The fluid pressure transducer 352 senses pressure of the fluid inside a “high side” fluid line and is connected to the data accumulator 350 and operates in conjunction therewith to provide a signal or data which is capable of being stored and read as meaningful data from the data accumulator assembly 350. As may be understood, such data can be “date-stamped”, or associated with a particular date and time, by known means via use of said clock 354.

Clock 354

The clock 354 provides date-stamping capability to signals stored in the data accumulator assembly 350. Preferably, most if not all data collected will be date stamped.

Foot Carriage Location Sensor 356

The carriage location sensor 356 senses the relative location of the foot support carriage assembly 30 and is connected to the data accumulator 350 and operates in conjunction therewith to provide a signal or data which is capable of being stored and read as meaningful data from the data accumulator assembly 350. As may be understood, such data can be “date-stamped”, or associated with a particular date and time, by known means via use of said clock 354.

There are many different ways to calculate the stroke of the foot support carriage assembly 30, including but not limited to the magnetic, infrared, or ultrasonic sensors as known in the art. The sensing could be done of the shaft of the hydraulic cylinder, or could be done of the carriage itself; it makes no difference assuming they both are traveling together and along the same linear axis.

Seat Sensor 358

A Seat Sensor 358 (such as a force pad or load cell) could be used which could either be a basic on/off type weight presence sensor (to recognize a weight over a given weight threshold) or alternatively if could be a weight sensor. This would allow the recordation of data relating to the sensing of the time the patient is actually in the seat of the device, as well as the weight of the patient if so desired. This would also allow a better determination of whether the patient remained in the chair or not between stretch periods. Such input could have advantages for gathering research data. As may be understood, such data can be “date-stamped”, or associated with a particular date and time, by known means via use of said clock 354.

Other Potential Inputs

It should be understood that other inputs are anticipated, including a high side fluid pressure switch (such as 252 identified above) separate from the transducer 352, which could be used for on/off capability of the device.

Instead of determining force exerted by the foot by measuring high side hydraulic pressure, instead a load sensor could be placed underneath the foot. This could be provided by a pressure sensing foot pad, a load cell, or the like.

Other inputs could include:
footplate pressure sensors
EMG or electromyographic sensor data of the muscle function
Seat or seat back pressure sensors
hand or grip control pressure sensors
video based measurement or compliance systems

Data Accumulator Assembly 350 Outputs

The following are potential outputs of the data accumulator assembly 350 of FIG. 17:
360 Antenna
362 Direct Download Port
363 Hard Wire Connection

Antenna 360

The antenna 360 is configured to transmit data wirelesslessly to a central data storage device 400 described later in this application. Any type of wireless transmission (cellular, radio, satellite) or the like is contemplated under one embodiment of the invention.

Direct Download Port

The direct download port 360 is configured to transmit data to a central data storage device 400 described later in this application. Such a port could facilitate any type of known “hard wire” type connection 363, including but not limited to USB, Firewire, Ethernet, cable or telephone modem, or the like. A “card reader/writer” or the like could also be included to read and write compatible portable media which can then be written to and detached for use elsewhere as described below.

Other Information Gathered to Accommodate Leg Length

It has been recognized that the ability to estimate the torque on the knee of a patient, as well as to determine the degrees of flexion of the knee, can be an important aspect of analyzing and treating the knee.

As noted above, data can be accumulated relating to the force being exerted by the device 300 on the leg of the patient. By calculation and/or empirical testing, the correspondence between fluid pressure being sensed by the data accumulator assembly 350 and the force being exerted on the foot of the patient can be determined.

However, a given amount of force applied by the foot support carriage assembly 30 can result in different torques on different knees, depending on associated leg dimensions. For a given position of the foot support carriage assembly 30, a shorter leg will experience less torque than a longer leg when encountering a given force by the foot support carriage assembly. A similar phenomena applies to the amount of flexion being experienced by the knee; for a given stroke distance of the foot support carriage assembly 30, a shorter leg will experience more flexion than a longer leg.

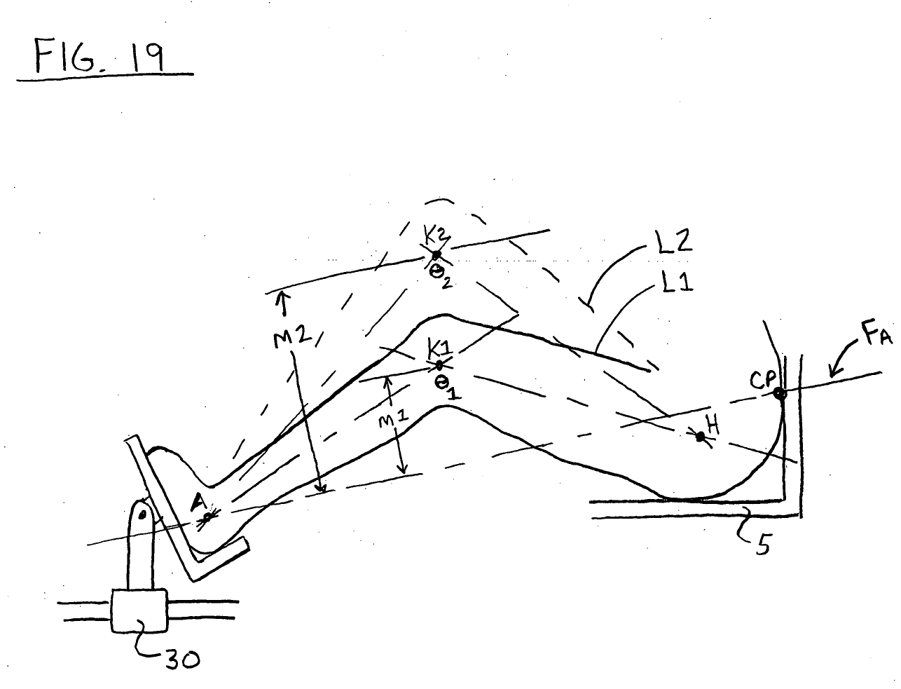

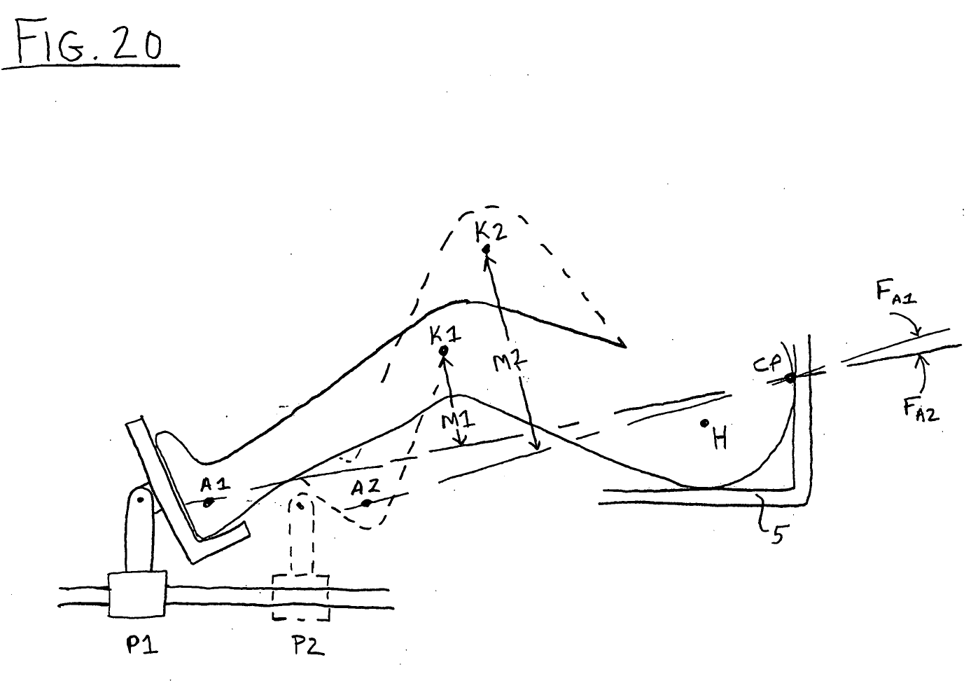

Reference is now made to FIG. 19, which is an illustrative view showing the relative torque and angular displacement encountered by two different legs, shorter leg L1 (shown in solid line) and longer leg L2 (shown in dotted line) for a given position of the foot support carriage assembly 30 and chair 5.

Leg L1 has a knee with an exemplary pivot point K1, a hip pivot point H, and an ankle pivot point A. For purpose of this discussion the length of the femur in leg L1 will be assumed to be the distance between H and K1, and the length of the tibia in leg L1 will be assumed to be the distance between K1 and A.

Leg L2 has a knee with an exemplary pivot point K2, a hip pivot point H, and an ankle pivot point A. For purpose of this discussion the length of the femur in leg L2 will be assumed to be the distance between H and K2, and the length of the tibia in leg L2 will be assumed to be the distance between K2 and A. As may be seen it is assumed for this discussion that the hip and ankle joints associated with the two legs are located at the same positions H and A, respectively.

When the carriage 30 moves towards the chair 5, eventually a force will be applied to the leg in the device. For each of the exemplary legs shown, this force will be assumed to be along force axis F_{A}, which passes between the ankle joint A and the point at which the lower back of the patient contacts the chair at CP.

As can be seen, for a given position of the foot support carriage assembly 30 and chair 5, the shorter leg L1 exhibits a knee bend angle Θ1 (THETA1) which is less than the knee bend angle Θ2 (THETA2) of leg L2, that is, the knee joint K2 is “bent” less than knee joint K1. It will be understood that for a given stroke distance of the support carriage assembly 30 along its linear path, assuming bending is occurring, the shorter leg will bend more, as it takes less stroke distance to do the same bending. Said another way, for a given distance of liner movement of the foot support carriage assembly 30, there is less angular displacement of the knee of a longer leg compared to the knee of a shorter leg. The inventors have discovered that there is value in this discovery, and there is also value in the using leg dimension data in addition to the other data mentioned in this application, in order to better analyze knee recovery by factoring in leg dimension data.

Continuing to refer to FIG. 19, it may also be seen that a similar analysis shows that for a given position of the foot support carriage assembly 30 and chair 5, and assuming a force vector along axis FA, the torque on the two legs include two different effective lever arm distances. In the case of leg L1, the lever (or “moment”) arm distance is M1; in the case of leg L1, the lever arm distance is M2. Thus for given force vector along axis F_{A}, when encountering a given force by the foot support carriage assembly 30 the knee of the longer leg L2 will be experiencing more torque than the knee of the shorter leg L1.

Assuming that the force vector along axis along axis F_{A} can be related to the fluid pressure being measured as noted above, the knee of the longer leg L2 will be experiencing more torque than the knee of the shorter leg L1 although the reading of high side fluid pressure will appear the same. The inventors have discovered that there is value in this discovery, and there is also value in the using leg dimension data in addition to the other data mentioned in this application, in order to better analyze knee recovery by factoring in leg dimension data.

Reference is now made to Figure H, which shows the leg of one patient at two different carriage positions P1 and P2, the first position P1 being further extended than the other (P2). Assuming there is force, as may be seen the force axis along axis F_{A1} at position P1, and along axis F_{A2} at position A2. The effective lever (or “moment”) arm distance at position P1 is M1, which as may be seen is less than the effective lever arm distance at position P2. M1 is the tangential distance of knee joint K1 from axis A1; M2 is the tangential distance of knee joint K2 from axis A2. As may be seen, as the carriage moves along a linear axis which does not include the contact point CP, the force axes such as A1 and A2 vary in orientation depending on the location of the carriage.

Therefore it may be seen that while fluid pressure and stroke distance readings are certainly valuable, without knowing the dimensions of the particular leg encountering the force, it can be difficult to most accurately determine the actual torque that the knee is encountering as well as its angular displacement. One embodiment of the present invention contemplates the gathering of leg dimension data specific to each patient in order to better measure such torque. Once the dimensions of the relevant leg of the patient are better known (given that carriage displacement and force on the leg data is known), such data can be used later to better analyze knee joint data.

Time and Manner of Taking Leg Dimension Readings

It has been recognized by the inventors that it would be advantageous to accumulate such leg dimension data at the same time the device (be it 10, 200 or 300) is being set up for use by a physical therapist or the like at the home of the patient or at the office of the physical therapist, although other times are also possible. As such, it may be recognized that it would be advantageous for any leg or other body measurements to be taken with the use of commonly available instruments such as a tape measure, goniometer or the like, using measuring techniques as known in the art.

The inventors have recognized that there are different manners in which this leg dimension data could be accumulated.

One manner would be by simply measuring the height of the patient and applying an empirically derived factor to approximate the needed leg measurement data.

Another manner would be by making leg-specific measurements, such as measurements of the length of femur and the length of the tibia. One way to measure such dimensions would be through the use of hip, knee, and ankle full length ex-rays. Manual measurement by a clinician using a ruler or goniometer or the like could also be used.

Once the leg dimensions are known, known measurement, geometric trigometric, and other mathematical principles can be used to approximate the movement of the leg in light of the measured movement of the foot support carriage assembly 30.

Data Point Storage Protocol

As noted above, the data accumulator assembly 350 is capable of storing data relating to conditions sensed by the fluid pressure transducer 352, the clock 354, the carriage position sensor 356, and the seat sensor 358.

As may be understood, the presence of multiple signal and/or data inputs allow for a vast majority of different ways the data could be stored. However, at the same time, it may not be feasible to store excessively detailed data corresponding to the location of the carriage and the pressure of the high side line down to the millisecond. This is due not only to digital storage limitations due to the additional data required, but also power limitations should the device be battery powered. As may be understood, the various sensors used each can require power to operate, and thus in some cases the less readings taken, the less power needed.

Therefore the following data point gathering protocols are contemplated under the present invention:
Time-stamped pressure and carriage displacement data every 5 seconds (or 1 second or less, but some regular time increment).
Recording of any peak loads or distances, such as peak pressures encountered, or peak travel distances, independent of any periodically gathered data such as noted in the paragraph above. The system could be set up to recognize peaks or could search for such peaks after the data is gathered.

The above protocols are for purpose of example only and should not be construed as limiting.

Conversion of Signals to Data

It should be understood that there are a wide variety of ways in which the signal from the various inputs to the data accumulator assembly 350 could provide such data, and how the data could be stored, and in what format the data could be. For example, the sensors could provide an analog signal output via a level of voltage or amperage, or could provide a digital output. In the case of analog input, some conversion as known in the art could be used. Appropriate conversion factors or the like can be used as known in the art to provide data which corresponds to the pressure and distances measured. As such data is passed along a path to its final destination, this conversion could be done at any desired point along the path (e.g., before or after it is sent from the data accumulator assembly 350 to the central data storage device 400 described below.

The data could be stored in the data accumulator assembly 350 via known “over-the-counter” devices as needed.

It should also be understood that if the system is hardwired to anything (internet, phone line, etc.) the data could be recorded remotely by the server and that recording link would be activated when the system turns on and not be a continuously open port.

Data Transmission Protocol

Data can be transferred from the data accumulator assembly 350 to an external source such as the central data storage device 400 described below by various means known in the art, such as by the use of wireless modems communicating through cellular or other wireless technology (see for example antenna 360 in FIG. 17), via a hookup to the internet (see direct download port 362 in FIG. 17) or through a direct data connection at the site of the central data storage device 400 (see direct download port 362 in FIG. 17), or the like.

Options could include connections to the home computers of patients, or hard line modem connections.

One possible preference would be for the device 350 to be battery powered and wirelessly operated. In this case cellular modems would likely be used.

Central Data Storage and Dissemination

One embodiment of the present invention contemplates the transmission of the data accumulated in the data accumulator assembly 350 of FIG. 17 to a “central” data storage facility in order that the data may be readily accessed through various means and by various entities.

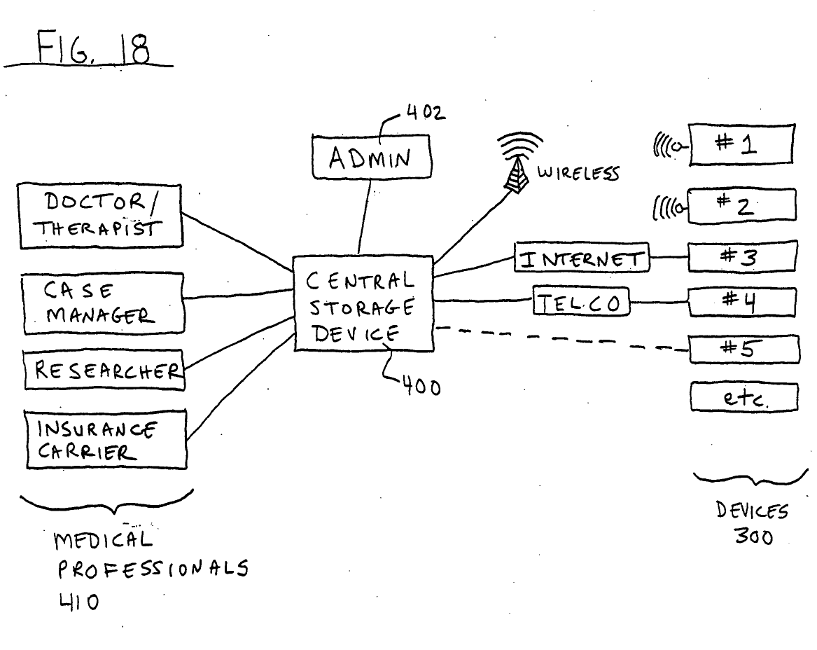

FIG. 18 shows a block diagram including the central storage device 400, an administrative user 402, various exemplary medical professionals 410, and exemplary Compliance Version Two apparatuses 300.

Central Storage Device 400

Reference is now again made to FIG. 18, which shows a central storage device 400, which is a device (which could include multiple separate components) for the storage and exchange of data relating to data being generated under the invention, through various means known in the art, which can include but is not limited to magnetic, optical, or other similar data storage through conventional computers or the like, as well as paper or other known storage media.

Medical Professionals 400

FIG. 18 shows exemplary users of the data of the central storage device 400, which in one embodiment include medical professionals (which can include physicians and their assistants, physical therapists, medical researchers, and insurance providers (e.g., case managers)). Under one embodiment of the present invention, said users are allowed access to the central storage device 400 as known in the art, including but not limited to the use of the internet via known browser-based technology, or other known electronic access means.

Administrator 402

The administrator 402 is provided access as needed by known administrative access means in order to both service the overall system as well as to manage and manipulate the data as needed.

Connection with Devices 300

FIG. 18 shows various exemplary Compliance Version Two apparatuses 300, which are provided with various connection capabilities relative to the central storage device 400, so as to provide data transfer thereto. Exemplary apparatuses #1 and #2 are shown with wireless connections. Exemplary apparatus #3 provides data transmission through an internet connection. Exemplary apparatus #4 provides data transmission through a telephone modem connection. Exemplary device #5 provides data transfer through either media transfer (e.g. a flash drive or other such data storage device such as the portable media described in connection with the direct download port 360 described above) or through a direct connection, again as described in connection with the direct download port described above.

Treatment Protocol Used With Version 2

The treatment protocol for Version 2 can be similar to that described with respect to that described above; a given patient is instructed to conduct multiple separate treatment sessions, as an example three sessions a day, with each session including a certain amount of stretch time (as an example twenty minutes), which does not include intermediate “relax” time. In this example, this provides one full hour of “stretch” and “relax” therapy.

As in the case of Version 1 this protocol of use by the patient could however also include the use of the readout panel 351. As noted above this panel provides a visually perceptible indication of the elapsed time (since last reset) that the pressure switch has been closed; the patient could use the data accumulator readout panel 351 to determine when the appropriate amount of “stretch” time has been accomplished, be it for the particular session, the three-session day, for the week, etc. This could even be a part of a prescribed order by a physician (e.g., patients are told to try to log at least 1 hour a day, etc.).

Data Output and Analysis of Version 2

Once the data as stored in the data accumulator assembly 350 is transferred to the central data storage device 400, it can then be accessed and analyzed by a variety of entities for various purposes. As described above, this data can include:
High side pressure readings (date stamped), or such reading converted to force readings
Carriage position readings (date stamped)
   Time in the machine (through use of the seat or some other sensor)
   Leg measurement readings (assuming taken)
   Time of Stretch

This data can be used for a variety of purposes, by a variety of different entities.

For example, a patient case manager could access the central storage device 400 to review the progress that a particular patient or groups of patients could be making. Known technology could be used to allow the patient case manager daily, weekly, or monthly, access to see the extent to which the patient sample is or has been using the device, as well as to see the extent that progress (improvement in angular displacement) is being made.

For example, it could be beneficial for an insurance case manager to see the usage of each of the thirty days elapsed, to see if the patients are using the device every day, only on work days but not on the weekend (or vice versa), how consistent the usage is, and how this relates to increase in motion (as increase in motion is the desired end result.)

The analysis of the data could be done in relation to outcomes scored using traditional outcome analyses, including those relating to:
SF-12
KOOS knee osteoarthritis outcomes score
Cincinnati knee score
Visual analog scale testing

Advantages of Version 2

The advantages of Version 2 include those in Version 1, namely to provide the provision of a readout (if so desired under the treatment protocol) for the patient to view elapsed stretch time according to predetermined criteria.

However, there are many additional advantages of Version 2 due to the additional data being gathered, stored, and downloaded to a separate site. This has the potential for providing vast improvements in patient treatment, not only for the patients using the devices, but for future patients which can benefit from improvements in treatment protocol.

Opportunities for Modifying Protocols as Well as Future Devices

As may be understood, the access to and use of the data above, gathered from either Versions 1 or 2, would assist in better understanding the types of usage protocols that tend to result in better or worse outcomes. Then it would be possible to learn from the data and then use that information to modify the protocol as needed.

For example, it could be possible to determine if certain uses (or abuses) of various protocols tend to work better than others. As the data is accumulated, it can not only be used to assist the patients currently using the devices, but historical data can be developed which can be used for research and analysis.

### CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1-6. (canceled)

7. A leg manipulating device for manipulating the leg of a user sitting in a seat and for measuring at least one characteristic of said leg during said manipulation, said device comprising:
an elongate frame member having a longitudinal axis and a first and a second end;
a foot support carriage member movably attached relative to said elongate frame member, said foot support carriage member including a foot accepting portion configured for accepting the placement of the foot of said leg of said user while said user is sitting in said seat,
a force member operably associated with said foot support carriage member configured to cause said foot support carriage member to move along said elongate frame member so as to cause said leg of said user to be bent from a first, lesser-bent, position to a second, greater-bent, position, in which said foot provides more force against said foot accepting portion when in said second position when compared to said first position; and
a sensing device configured to determine when said foot of said patient provides at least a predetermined force against said foot accepting portion between said first and second positions.

8. The leg manipulating device as claimed in claim 7, further comprising a data accumulating device operably associated with said sensing device for accumulating data relating to the elapsed time said foot of said patient provides at least said predetermined force against said foot accepting portion.

9. The leg manipulating device as claimed in claim 8, further comprising a display associated with said data accumulating device for displaying said elapsed time for view by said user while using said device.

10. The leg manipulating device as claimed in claim 9, wherein said force member includes a hydraulic cylinder in communication with hydraulic fluid, and wherein said sensing device includes a hydraulic sensor for sensing the hydraulic pressure within said fluid.

11. The leg manipulating device as claimed in claim 10, wherein said hydraulic sensor is configured to be set at a predetermined pressure such that a particular signal is delivered to said sensing device when said predetermined pressure is reached.

12. The leg manipulating device as claimed in claim 11, wherein said hydraulic cylinder is configured to be selectively pressurized via manual control by said user.

13. The leg manipulating device as claimed in claim 10, wherein said hydraulic cylinder is configured to be selectively pressurized via manual control by said user.

14. The leg manipulating device as claimed in claim 9, wherein said force member includes a hydraulic cylinder in communication with hydraulic fluid, and wherein said sensing device includes a hydraulic sensor for sensing the hydraulic pressure within said fluid.

15. The leg manipulating device as claimed in claim 8, wherein said sensing device includes a load cell associated with said foot accepting portion.

16. The leg manipulating device as claimed in claim 7, wherein said force member includes a hydraulic cylinder in communication with hydraulic fluid, and wherein said sensing device includes a hydraulic sensor for sensing the hydraulic pressure within said fluid.

17. The leg manipulating device as claimed in claim 16, wherein said hydraulic sensor is configured to be set at a predetermined pressure such that a particular signal is delivered to said sensing device when said predetermined pressure is reached.

18. The leg manipulating device as claimed in claim 17, wherein said hydraulic cylinder is configured to be selectively pressurized via manual control by said user.

19. The leg manipulating device as claimed in claim 16, wherein said hydraulic cylinder is configured to be selectively pressurized via manual control by said user.

20. The leg manipulating device as claimed in claim 7, wherein said sensing device includes a load cell associated with said foot accepting portion.

21. A method for manipulating the leg of a user having a posterior suitable for sitting in a seat, said method comprising the steps of:
A) providing a leg manipulating device including the following associated elements:
1) an elongate frame member having a longitudinal axis and a first and a second end;
2) a foot support carriage member movably attached relative to said elongate frame member, said foot support carriage member including a foot accepting portion configured for accepting the placement of the foot of said leg of said user while said user is sitting in said seat,
3) a force member operably associated with said foot support carriage member, said force member configured to cause said foot support carriage member to move along said elongate frame member; and
4) a sensing device configured to determine when said foot of said patient provides at least a predetermined force against said foot accepting portion between said first and second positions;
B) situating the posterior of said user in said seat;
C) operating said force member such that foot support carriage member moves along said elongate frame member so as to cause said foot support carriage member to move along said elongate frame member so as to cause said leg of said user to be bent from a first, lesser-bent, position to a second, greater-bent, position, in which said foot provides more force against said foot accepting portion when in said second position when compared to said first position; and
D) measuring at least one characteristic of said leg during at least a portion of step “C” by use of said sensing device.

22. The leg manipulating method as claimed in claim 21, wherein in step “D”, a data accumulating device operably associated with said sensing device is used for accumulating data relating to the elapsed time said foot of said patient provides at least said predetermined force against said foot accepting portion.

23. The leg manipulating method as claimed in claim 22, wherein in step “D”, a display associated with said data accumulating device is used for displaying said elapsed time for view by said user while using said device, and wherein in step “D”, said user determines the extent of use of said leg manipulating device upon review of said elapsed time on said display.

24. The leg manipulating method as claimed in claim 23, wherein in Step “A”, said force member includes a hydraulic cylinder, and wherein said sensing device includes a hydraulic sensor for sensing the hydraulic pressure within fluid in communication with said cylinder, said sensor set at a predetermined pressure so as to provide a particular signal to said sensing device when said predetermined pressure is reached, and wherein in Step “C”, said hydraulic cylinder is selectively pressurized via manual control by said user.

25. The leg manipulating method as claimed in claim 23, wherein in Step “A”, said sensing device includes a load cell associated with said foot accepting portion, and wherein in Step “D”, said measurement at least one characteristic of said leg during at least a portion of step “C” includes the use of said load cell.

26. The leg manipulating method as claimed in claim 22, wherein in Step “A”, said force member includes a hydraulic cylinder, and wherein said sensing device includes a hydraulic sensor for sensing the hydraulic pressure within fluid in communication with said cylinder, said sensor set at a predetermined pressure so as to provide a particular signal to said sensing device when said predetermined pressure is reached, and wherein in Step “C”, said hydraulic cylinder is selectively pressurized via manual control by said user.

27. The leg manipulating method as claimed in claim 22, wherein in Step “A”, said sensing device includes a load cell associated with said foot accepting portion, and wherein in Step “D”, said measurement at least one characteristic of said leg during at least a portion of step “C” includes the use of said load cell.

28. A method for manipulating the leg of a user having a posterior suitable for sitting in a seat, said method comprising the steps of:
A) providing a leg manipulating device including the following associated elements:
1) an elongate frame member having a longitudinal axis and a first and a second end;
2) a foot support carriage member movably attached relative to said elongate frame member, said foot support carriage member including a foot accepting portion configured for accepting the placement of the foot of said leg of said user while said user is sitting in said seat,
3) a force member operably associated with said foot support carriage member, said force member configured to cause said foot support carriage member to move along said elongate frame member; and
4) a sensing device configured to determine when said foot of said patient provides at least a predetermined force against said foot accepting portion between said first and second positions; and
B) situating the posterior of said user in said seat;
C) operating said force member such that foot support carriage member moves along a first direction relative to said elongate frame member so as to cause said foot support carriage member to move along said elongate frame member so as to cause said leg of said user to be bent from a first, lesser-bent, position to a second, greater-bent, position, in which said foot provides more force against said foot accepting portion when in said second position when compared to said first position;
D) measuring at least one characteristic of said leg during at least a portion of step “C” by use of said sensing device;
E) operating said force member such that foot support carriage member moves along a second direction relative to said elongate frame member, said second direction being opposite to said first direction, so as to cause said foot support carriage member to move along said elongate frame member so as to cause said leg of said user to be bent from said second, greater-bent, position, to a third, intermediate-bent, position in which said foot provides less force against said foot accepting portion when compared to said second position, but greater force when compared to said first position; and
F) measuring the said same one characteristic of said leg measured at step “D”, during at least a portion of step “E” by use of said sensing device.

29. The leg manipulating method as claimed in claim 28, wherein during steps “D” and “E”, a data accumulating device operably associated with said sensing device is used for accumulating data relating to the elapsed time said foot of said patient provides at least said predetermined force against said foot accepting portion.

30. The leg manipulating method as claimed in claim 28, wherein during at least one of steps “D” and “E”, said foot member is moving relative to said elongate frame member during said measurement of said one characteristic of said leg.

31. The leg manipulating method as claimed in claim 28, wherein during at least one of steps “D” and “E”, said foot member is moving relative to said elongate frame member during said measurement of said one characteristic of said leg.

32. A method for manipulating the leg of a user having a posterior suitable for sitting in a seat, said method comprising the steps of:
A) providing a leg manipulating device including the following associated elements:
1) a frame member;
2) a foot support carriage member movably attached relative to said frame member along an axis, said foot support carriage member including a foot accepting portion configured for accepting the placement of the foot of said leg of said user,
3) a force member operably associated with said foot support carriage member, said force member configured to cause said foot support carriage member to move along said axis relative to said frame member; and
4) a sensing device configured to determine when said foot of said patient provides at least a predetermined force against said foot accepting portion between said first and second positions; and
5) a data accumulating device configured for accumulating data relating to the elapsed time said foot of said patient provides at least said predetermined force against said foot accepting portion,
B) situating the posterior of said user in said seat;
C) operating said force member such that foot support carriage member moves along a first direction relative to said frame member so as to cause said foot support carriage member to move along said frame member so as to cause said leg of said user to be bent from a first, lesser-bent, position to a second, greater-bent, position, in which said foot provides more force against said foot accepting portion when in said second position when compared to said first position, said force being greater than said predetermined force;
D) measuring at least one characteristic of said leg during at least a portion of step “C” by use of said sensing device;
E) accumulating data during step “D” by use of said data accumulating device, relating to the elapsed time said foot of said patient provides at least said predetermined force against said foot accepting portion;
F) operating said force member such that foot support carriage member moves along a second direction relative to said frame member, said second direction being opposite to said first direction, so as to cause said foot support carriage member to move along said frame member so as to cause said leg of said user to be bent from said second, greater-bent, position, to a third, intermediate-bent, position in which said foot provides less force against said foot accepting portion when compared to said second position, said less force being less than said predetermined force;
G) measuring the said same one characteristic of said leg measured at step “D”, during at least a portion of step “F” by use of said sensing device;
H) accumulating data during step “G” by use of said data accumulating device, relating to the elapsed time said foot of said patient provides at least said predetermined force against said foot accepting portion, said data accumulation being done prior to the point at which said foot provides a force against said foot accepting portion being less than said predetermined force;
I) operating said force member such that foot support carriage member moves along said first direction relative to said frame member, so as to cause said foot support carriage member to move along said frame member so as to cause said leg of said user to be bent from said third position to a fourth position in which said foot provides greater force against said foot accepting portion when compared to said third position, said greater force being greater than said predetermined force;
J) measuring the said same one characteristic of said leg measured at step “D”, during at least a portion of step “F” by use of said sensing device;
K) accumulating data during step “G” by use of said data accumulating device, relating to the elapsed time said foot of said patient provides at least said predetermined force against said foot accepting portion, said data accumulation being done after the point at which said foot provides a force against said foot accepting portion being greater than said predetermined force; and
L) combining the data accumulated during steps E, H, and K.

33. The leg manipulating method as claimed in claim 32, wherein in Step “A”, said force member includes a hydraulic cylinder, and wherein said sensing device includes a hydraulic sensor for sensing the hydraulic pressure within fluid in communication with said cylinder, said sensor set at a predetermined pressure so as to provide a particular signal to said sensing device when said predetermined pressure is reached, and wherein in Step “C”, said hydraulic cylinder is selectively pressurized via manual control by said user.

34. The leg manipulating method as claimed in claim 33, wherein in Step “A”, said fluid in communication with said cylinder includes a pressure relief device, and wherein in Step “C”, said pressure relief device is selectively activated via manual control by said user.

35. The leg manipulating method as claimed in claim 32, wherein in Step “A”, said sensing device includes a load cell associated with said foot accepting portion, and wherein in Step “D”, said measurement at least one characteristic of said leg during at least a portion of step “C” includes the use of said load cell.

36. The leg manipulating method as claimed in claim 32, wherein in Step “A”, a display associated with said data accumulating device is used for displaying said elapsed time for view by said user while using said device, and wherein in step “D”, said user determines the extent of use of said leg manipulating device upon review of said elapsed time on said display.

37. A method for manipulating the leg of a user, said method comprising the steps of:
A) providing a leg manipulating device including the following associated elements:
1) a frame member;
2) a foot support carriage member movably attached relative to said frame member along an axis, said foot support carriage member including a foot accepting portion configured for accepting the placement of the foot of said leg of said user,
3) a force member operably associated with said foot support carriage member, said force member configured to cause said foot support carriage member to move along said axis relative to said frame member; and
4) a sensing device configured to determine when said foot of said patient provides at least a predetermined force against said foot accepting portion between said first and second positions; and
5) a data accumulating device configured for accumulating data relating to the elapsed time said foot of said patient provides at least said predetermined force against said foot accepting portion,
B) situating the posterior of said user relative to said device;
C) operating said force member such that foot support carriage member moves along said axis so as to cause said leg of said user to be bent in a plurality of positions, including a plurality of relatively greater-bent positions in which said foot of said patient provides a force being greater than said predetermined force, and also including a plurality of relatively lesser-bent positions in which said foot of said patient provides a force being lesser than said predetermined force, such that a during said operation said foot;
D) measuring at least one characteristic of said leg during at least a portion of step “C” by use of said sensing device; and
E) accumulating data during step “D” by use of said data accumulating device, relating to the total elapsed time said foot of said patient provides at least said predetermined force against said foot accepting portion during Step “C”.

38. The leg manipulating method as claimed in claim 37, wherein in Step “A”, said force member includes a hydraulic cylinder, and wherein said sensing device includes a hydraulic sensor for sensing the hydraulic pressure within fluid in communication with said cylinder, said sensor set at a predetermined pressure so as to provide a particular signal to said sensing device when said predetermined pressure is reached, and wherein in Step “C”, said hydraulic cylinder is selectively pressurized via manual control by said user so as to cause leg of said user to be bent from one of said lesser-bent positions to one of said greater-bent positions.

39. The leg manipulating method as claimed in claim 38, wherein in Step “A”, said fluid in communication with said cylinder includes a pressure relief device, and wherein in Step “C”, said pressure relief device is selectively activated via manual control by said user so as to cause leg of said user to be bent from one of said greater-bent positions to one of said lesser-bent positions.

40. The leg manipulating method as claimed in claim 37, wherein in Step “A”, said sensing device includes a load cell associated with said foot accepting portion, and wherein in Step “D”, said measurement at least one characteristic of said leg during at least a portion of step “C” includes the use of said load cell.

41. The leg manipulating method as claimed in claim 37, wherein in Step “A”, a display associated with said data accumulating device is used for displaying said elapsed time for view by said user while using said device, and wherein in step “D”, said user determines the extent of use of said leg manipulating device upon review of said elapsed time on said display.

42. A leg manipulating device for manipulating the leg of a user sitting in a seat and for measuring at least one characteristic of said leg during said manipulation, said device comprising:
A) a frame member;
B) a foot support carriage member movably attached relative to said frame member along an axis, said foot support carriage member including a foot accepting portion configured for accepting the placement of the foot of said leg of said user,
C) a force member operably associated with said foot support carriage member, said force member configured to cause said foot support carriage member to move along said axis relative to said frame member; and
D) a sensing device configured to determine when said foot of said patient provides at least a predetermined force against said foot accepting portion between said first and second positions; and
E) a data accumulating device configured for accumulating data relating to the elapsed time said foot of said patient provides at least said predetermined force against said foot accepting portion.

43. The leg manipulating method as claimed in claim 42, wherein said sensing device of element “D” comprises a fluid pressure transducer.

44. The leg manipulating method as claimed in claim 42, wherein said data accumulating device of element “E” includes a clock.

45. The leg manipulating method as claimed in claim 44, wherein said data accumulating device of element “E” includes a time stamping capability based on interaction with said clock.

46. The leg manipulating method as claimed in claim 42, further comprising:
F) a foot carriage location sensor.

47. The leg manipulating method as claimed in claim 46, wherein said foot carriage location sensor includes at least one of magnetic-, infrared-, or ultrasonic-based measurement.

48. The leg manipulating method as claimed in claim 42, further comprising:
F) a seat for accepting the posterior of a user; and
G) a seat sensor for sensing when a user is positioned within said seat.

49. The leg manipulating method as claimed in claim 48, wherein said seat sensor includes at least one of a force pad or load cell.

50. The leg manipulating method as claimed in claim 48, wherein said seat includes a seat back, and further comprising:
H) a back sensor for sensing the back of a user positioned within said seat.

51. The leg manipulating method as claimed in claim 42, further comprising:
F) a data output link configured to facilitate the transmission of data accumulated via said data accumulating device to an external location

52. The leg manipulating method as claimed in claim 51, wherein said data output link includes at least one of the following: antenna, direct download port, hard wire connection.

53. The leg manipulating method as claimed in claim 42, further comprising:
F) a high side fluid pressure switch associated with an on/off switch.

54. A method for manipulating the leg of a user, said method comprising the steps of:
A) providing a leg manipulating device including the following associated elements:
1) a frame member;
2) a foot support carriage member movably attached relative to said frame member along an axis, said foot support carriage member including a foot accepting portion configured for accepting the placement of the foot of said leg of said user,
3) a force member operably associated with said foot support carriage member, said force member configured to cause said foot support carriage member to move along said axis relative to said frame member; and
4) a sensing device configured to determine when said knee encounters bending; and
5) a data accumulating device configured for accumulating data relating to the elapsed time said knee encounters a predetermined level of bending,
B) situating the posterior of said user relative to said device;
C) operating said force member such that foot support carriage member moves along said axis so as to cause said leg of said user to be bent in a plurality of positions;
D) measuring the bending of said knee during at least a portion of step “C” by use of said sensing device; and
E) accumulating data during step “D” by use of said data accumulating device, relating to the total elapsed time said knee of said patient encounters at least said predetermined level of bending.

55. The method for manipulating the leg of a user as claimed in claim 54, wherein step “E” includes the accumulation of data relating to peak knee bending levels.

56. The method for manipulating the leg of a user as claimed in claim 54, wherein step “E” includes the accumulation of data relating to the time the user is positioned in the device.

57. A system for disseminating information relating to the manipulation of the leg of a user, said system including:
A) a leg manipulating device including the following associated elements:
1) a frame member;
2) a foot support carriage member movably attached relative to said frame member along an axis, said foot support carriage member including a foot accepting portion configured for accepting the placement of the foot of said leg of said user,
3) a force member operably associated with said foot support carriage member, said force member configured to cause said foot support carriage member to move along said axis relative to said frame member; and
4) a sensing device configured to determine when said knee encounters bending; and
5) a data accumulating device configured for accumulating data relating to the elapsed time said knee encounters a predetermined level of bending; and
B) a device for disseminating said data from step A-5 to a location remote from said device.
